(19) United States
(12) Patent Application Publication
(10) Pub. No.: US 2003/0134404 A1
(43) Pub. Date: Jul. 17, 2003

(54) Methods for producing stocks of recombinant AAV virions

(76) Inventors: Michael A. Lochrie, Hayward, CA (US); Peter Colosi, Alameda, CA (US)

Correspondence Address: COOLEY GODWARD LLP (R & P), FIVE PALO ALTO SQUARE, 3000 EL CAMINO REAL, PALO ALTO, CA, 94306-2155, US

(21) Appl. No.: 10/304,828

(22) Filed: Nov. 26, 2002

Related U.S. Application Data
(60) Provisional application No. 60/333,445, filed on Nov. 26, 2001.

(51) Int. Cl. C12N 7/02

(52) U.S. Cl. 435/239

(57) ABSTRACT

Methods for removing empty capsids from stocks of AAV virions comprising mixtures of empty and packaged capsids are described. The methods entail heating and adjusting the pH value of the stock, optionally in the presence of one or more chemical destabilizing agents.

### CROSS-REFERENCE TO RELATED APPLICATION

This application is related to provisional patent application serial No. 60/333,445, filed Nov. 26, 2001 from which application priority is claimed under 35 USC §119(e)(1) and which application is incorporated herein by reference in its entirety.

### TECHNICAL FIELD

The invention relates to methods for purifying adeno-associated virus (AAV) virions. More particularly, the invention relates to methods for purifying recombinant AAV (rAAV) virions containing packaged genomes from mixtures of AAV virions containing both packaged rAAV virions and empty AAV capsids lacking said genomes.

### BACKGROUND OF THE INVENTION

Scientists are continually discovering genes that are associated with human diseases such as diabetes, hemophilia, and cancer. Research efforts have also uncovered genes, such as erythropoietin, that are not associated with genetic disorders but instead code for proteins that can be used to treat numerous diseases. Despite significant progress in the effort to identify and isolate genes, however, a major obstacle facing the biopharmaceutical industry is how to safely and persistently deliver therapeutically effective quantities of gene products to patients.

Generally, the protein products of these genes are synthesized in cultured bacterial, yeast, insect, mammalian, or other cells and delivered to patients by direct injection. Injection of recombinant proteins has been successful but suffers from several drawbacks. For example, patients often require weekly, and sometimes daily, injections in order to maintain the necessary levels of the protein in the blood stream. Even then, the concentration of protein is not maintained at physiological levels. In particular, the level of the protein is usually abnormally high immediately following the injection, and far below optimal levels prior to the injection. Additionally, injection of recombinant protein often cannot deliver the protein to the target cells, tissues, or organs in the body. If the protein does reach its target, it is often diluted to non-therapeutic levels. Furthermore, the method is inconvenient and severely restricts the patient's lifestyle. The adverse impact on lifestyle is especially significant when the patient is a child.

These shortcomings have led to the development of gene therapy methods for delivering sustained levels of specific proteins into patients. These methods are designed to allow clinicians to introduce deoxyribonucleic acid (DNA) coding for a nucleotide sequence of interest directly into a patient (in vivo gene therapy) or into cells isolated from a patient or a donor (ex vivo gene therapy), which are subsequently returned to the patient. The introduced DNA then directs the patient's own cells or grafted cells to produce the desired protein product. Gene delivery, therefore, obviates the need for frequent injections. Gene therapy also allows clinicians to select specific organs or cellular targets (e.g., muscle, blood cells, brain cells, etc.) for therapy.

DNA may be introduced into a patient's cells in several ways. There are transfection methods, including chemical methods such as calcium phosphate precipitation and liposome-mediated transfection, and physical methods such as electroporation. In general, transfection methods are not suitable for in vivo gene delivery. There are also methods that use recombinant viruses. Current viral-mediated gene delivery vectors include those based on retrovirus, adenovirus, herpesvirus, pox virus, and adeno-associated virus (AAV). Like the retroviruses, and unlike adenovirus, AAV has the ability to integrate its genome into a host cell chromosome. Because of the unique features of viral-mediated gene transfer, the vast majority of gene therapy trials conducted have used viral-mediated gene delivery for their method of gene insertion. Hodgson, C. P. Biotechnology (1995) 13: 222-225.

AAV, a parvovirus belonging to the genus Dependovirus, has several attractive features not found in other viruses. For example, AAV can infect a wide range of host cells, including non-dividing cells. Furthermore, AAV can infect cells from different species. Importantly, AAV has not been associated with any human or animal disease, and does not appear to alter the physiological properties of the host cell upon integration. Finally, AAV is stable at a wide range of physical and chemical conditions, which lends itself to production, storage, and transportation requirements.

The AAV genome, a linear, single-stranded DNA molecule containing approximately 4700 nucleotides (the AAV-2 genome consists of 4681 nucleotides), generally comprises an internal non-repeating segment flanked on each end by inverted terminal repeats (ITRs). The ITRs are approximately 145 nucleotides in length (AAV-1 has ITRs of 143 nucleotides) and have multiple functions, including serving as origins of replication, and as packaging signals for the viral genome.

The internal non-repeated portion of the genome includes two large open reading frames (ORFs), known as the AAV replication (rep) and capsid (cap) regions. These ORFs encode replication and capsid gene products, respectively: replication and capsid gene products (i.e., proteins) allow for the replication, assembly, and packaging of a complete AAV virion. More specifically, a family of at least four viral proteins are expressed from the AAV rep region: Rep 78, Rep 68, Rep 52, and Rep 40, all of which are named for their apparent molecular weights. The AAV cap region encodes at least three proteins: VP1, VP2, and VP3.

AAV is a helper-dependent virus, requiring co-infection with a helper virus (e.g., adenovirus, herpesvirus, or vaccinia virus) in order to form functionally complete AAV virions. In the absence of co-infection with a helper virus, AAV establishes a latent state in which the viral genome inserts into a host cell chromosome or exists in an episomal form, but infectious virions are not produced. Subsequent infection by a helper virus “rescues” the integrated genome, allowing it to be replicated and packaged into viral capsids, thereby reconstituting the infectious virion. While AAV can infect cells from different species, the helper virus must be of the same species as the host cell. Thus, for example, human AAV will replicate in canine cells that have been co-infected with a canine adenovirus.

To take advantage of the many potential benefits of gene therapy using rAAV technology, the virions, containing the heterologous gene, must be successfully produced. To accomplish this, a suitable host cell line is transfected with an AAV vector containing a heterologous gene, but lacking rep and cap. AAV helper function genes (i.e., rep and cap) and accessory function genes are provided in separate vectors. Helper and accessory function gene products are expressed in the host cell where they act in trans on the rAAV vector containing the heterologous gene. The heterologous gene is then replicated and packaged as though it were a wild-type (wt) AAV genome, forming a recombinant virion.

After culturing the host cells with the necessary components for rAAV production, the host cell is harvested and a crude extract is produced. The resulting preparation will contain, among other components, AAV capsids with genomes containing the heterologous gene (i.e., “packaged capsids”) and AAV capsids lacking genomes (i.e., “empty capsids”). By some accounts, empty capsids can comprise as much as 80% of the AAV material found in the crude cell extract (see, for example, Grimm et al. (1999) Gene Ther 6:1322-30). Current laboratory purification techniques, such as cesium chloride density gradient centrifugation, are capable of separating empty capsids from packaged ones, but these techniques are not amenable to commercial-scale purification efforts.

In response to the need to generate large quantities of rAAV virions for commercial production, scalable purification techniques have been recently developed. Based on column chromatographic separation, the application of these techniques has resulted in the production of much larger amounts of rAAV, thereby facilitating the goal of generating sufficient quantities of rAAV to support the commercialization of rAAV gene therapy products. As mentioned above, however, more than 80% of AAV material created during rAAV production may be empty capsids, and current column chromatography purification techniques do not separate packaged capsids from empty capsids. There remains a need to discover new ways of distinguishing between empty and packaged capsids in the rAAV virion production and purification process. More particularly, there remains a need to eliminate or reduce the numbers of empty capsids from stocks of packaged capsids so that manufacturing capability is enhanced.

### SUMMARY OF THE INVENTION

The present invention is based on the discovery of efficient and commercially viable methods for producing stocks of rAAV virions with reduced amounts of empty capsids.

Accordingly, in one embodiment, the subject invention is directed to a method of reducing the number of empty capsids in purified stocks of AAV virions, with minimal loss to packaged capsids contained therein. The invention contemplates the use of the disclosed methods regardless of the process in which rAAV virions are produced. In certain preferred embodiments, AAV stocks are generated without co-infection by a helper virus, e.g., in a host cell line via triple-transfection with an accessory function vector, an AAV vector, and an AAV helper vector. After harvesting the transfected host cell, a lysate is formed by disrupting the transfected host cells using techniques suitable for large-scale production, filtering the recovered lysate, and purifying it using column chromatography.

In certain embodiments, the methods include treating the purified AAV stock with destabilizing agents, subjecting the stock to changes in pH, and heating the stock to preferentially denature empty capsids while maintaining the viability of packaged capsids. These steps can occur in any order.

AAV stocks are subjected to pH values from about 4.0 to 7.0, preferably from about 4.5 to 6.0, more preferably from about 4.5 to 5.5, and most preferably about 4.5 to about 5.0. The AAV stock is heated to temperatures from about 40-70° C., such as 40-60° C., 40-55° C., 40-50° C., 45-50° C., 45-65° C., 45-60° C., 50-70° C., 55-65° C., 60° C., or any temperature within these ranges.

Accordingly, in particular embodiments, the invention is directed to a method for removing empty AAV capsids from a mixture of AAV virions comprising empty and packaged AAV capsids. The method comprises:

heating the mixture to a temperature of between 40° C. and 70° C., such as 40-60° C., 40-55° C., 40-50° C., 45-50° C., 45-65° C., 45-60° C., 50-70° C., 55-65° C., 60° C., or any temperature within these ranges.

adjusting the pH value of the mixture to a pH between 4 and 7, such as between 4.0 and 5.5, e.g., about 4.5 to 5.

In certain embodiments, the mixture is heated for at least 4 to 5 minutes, such as for 4 or 5 to 10 or 20 minutes.

In additional embodiments, the method further comprises adding a chemical destabilizing agent to the mixture, such as SDS. The SDS may be present in the mixture at a concentration of between about 1% and 2%. In other embodiments, the chemical destabilizing agent is urea. The urea may be present at a concentration of between 3 molar and 8 molar, e.g., between 4 molar and 5 molar, such as about 4 molar. In certain embodiments, both SDS and urea are added to the mixture.

In still a further embodiment, the invention is directed to a method for removing empty AAV capsids from a mixture of AAV virions comprising empty and packaged AAV capsids. The method comprises:

heating the mixture for about 5-10 minutes to a temperature of between about 55° C. and about 65° C.;

adjusting the pH value of the mixture to a pH between about 4.0 and 5.5; and

adding one or more chemical destabilizing agents to the mixture.

In certain embodiments, the chemical destabilizing agent is SDS present in the mixture at a concentration of between about 1% and 2%. In other embodiments, the chemical destabilizing agent is urea. The urea may be present at a concentration of between 3 molar and 8 molar, e.g., between 4 molar and 5 molar, such as about 4 molar. In other embodiments, both SDS and urea are added to the mixture.

In the embodiments described above, heating and adjusting the pH may be done substantially concurrently. Alternatively, the mixture may be heated prior to or subsequent to adjusting the pH value.

In additional embodiments, the mixture is from a cell lysate obtained from cells rendered capable of producing AAV virions. The mixture can be obtained from a chromatographic column elution of the cell lysate.

In other embodiments, the method produces a stock of rAAV virions substantially free of empty AAV capsids, such as a stock wherein at least 75% to about 99% or more of the AAV virions present in the stock are packaged AAV capsids.

The methods provide for the testing of denatured capsids, including subjecting the treated AAV stock to SDS-polyacrylamide gel electrophoresis, then running the gel until sample material is separated, and blotting the gel onto membranes. Anti-AAV capsid antibodies are then used as the primary antibodies that bind to denatured capsid proteins. A secondary antibody is then used, one that binds to the primary antibody and contains a means for detecting binding with the primary antibody. A method for detecting binding is used to semi-quantitatively determine binding between the primary and secondary antibodies.

To test for infectious titer, the methods include the seeding of host cells into tissue culture-treated plates and incubating the cells for about 24 hours. Adenovirus and treated AAV stock is then added to the host cells. The host cells, adenovirus, and AAV stock are allowed to incubate for 24 hours, after which the host cells are fixed and stained with an appropriate agent that will detect the rAAV expressed transgene.

These and other embodiments of the subject invention will readily occur to those of skill in the art in view of the disclosure herein.

### BRIEF DESCRIPTION OF THE FIGURES

FIG. 1 depicts the result of an experiment in which mixtures of AAV virions (empty and packaged) were treated with 30 mM sodium acetate, pH 4.8, 4M urea and 1% SDS over a temperature range of 40-80° C.

### DETAILED DESCRIPTION OF THE INVENTION

The practice of the present invention will employ, unless otherwise indicated, conventional methods of virology, microbiology, molecular biology and recombinant DNA techniques within the skill of the art. Such techniques are explained fully in the literature. See, e.g., Sambrook et al. Molecular Cloning: A Laboratory Manual (Current Edition); DNA Cloning: A Practical Approach, Vol. I & II (D. Glover, ed.); Oligonucleotide Synthesis (N. Gait, ed., Current Edition); Nucleic Acid Hybridization (B. Hames & S. Higgins, eds., Current Edition); Transcription and Translation (B. Hames & S. Higgins, eds., Current Edition); CRC Handbook of Parvoviruses, vol. I & II (P. Tijssen, ed.); Fundamental Virology, 2nd Edition, vol. I & II (B. N. Fields and D. M. Knipe, eds.); Freshney Culture of Animal Cells, A Manual of Basic Technique (Wiley-Liss, Third Edition); and Ausubel et al. (1991) Current Protocols in Molecular Biology (Wiley Interscience, NY).

All publications, patents and patent applications cited herein, whether supra or infra, are hereby incorporated by reference in their entirety.

As used in this specification and the appended claims, the singular forms “a,” “an” and “the” include plural references unless the content clearly dictates otherwise.

A. Definitions

In describing the present invention, the following terms will be employed, and are intended to be defined as indicated below.

By “vector” is meant any genetic element, such as a plasmid, phage, transposon, cosmid, chromosome, virus, virion, etc., which is capable of replication when associated with the proper control elements and which can transfer gene sequences between cells. Thus, the term includes cloning and expression vehicles, as well as viral vectors.

By an “AAV vector” is meant a vector derived from an adeno-associated virus serotype, including without limitation, AAV-1, AAV-2, AAV-3, AAV-4, AAV-5, AAV-6, AAV-7 and AAV-8. AAV vectors can have one or more of the AAV wild-type genes deleted in whole or part, preferably the rep and/or cap genes, but retain functional flanking ITR sequences. Functional ITR sequences are necessary for the rescue, replication and packaging of the AAV virion. Thus, an AAV vector is defined herein to include at least those sequences required in cis for replication and packaging (e.g., functional ITRs) of the virus. The ITRs need not be the wild-type nucleotide sequences, and may be altered, e.g., by the insertion, deletion or substitution of nucleotides, so long as the sequences provide for functional rescue, replication and packaging.

“AAV helper functions” refer to AAV-derived coding sequences which can be expressed to provide AAV gene products that, in turn, function in trans for productive AAV replication. Thus, AAV helper functions include both of the major AAV open reading frames (ORFs), rep and cap. The Rep expression products have been shown to possess many functions, including, among others: recognition, binding and nicking of the AAV origin of DNA replication; DNA helicase activity; and modulation of transcription from AAV (or other heterologous) promoters. The Cap expression products supply necessary packaging functions. AAV helper functions are used herein to complement AAV functions in trans that are missing from AAV vectors.

The term “AAV helper construct” refers generally to a nucleic acid molecule that includes nucleotide sequences providing AAV functions deleted from an AAV vector which is to be used to produce a transducing vector for delivery of a nucleotide sequence of interest. AAV helper constructs are commonly used to provide transient expression of AAV rep and/or cap genes to complement missing AAV functions that are necessary for lytic AAV replication; however, helper constructs lack AAV ITRs and can neither replicate nor package themselves. AAV helper constructs can be in the form of a plasmid, phage, transposon, cosmid, virus, or virion. A number of AAV helper constructs have been described, such as the commonly used plasmids pAAV/Ad and pIM29+45 which encode both Rep and Cap expression products. See, e.g., Samulski et al. (1989) J. Virol. 63:3822-3828; and McCarty et al. (1991) J. Virol. 65:2936-2945. A number of other vectors have been described which encode Rep and/or Cap expression products. See, e.g., U.S. Pat. Nos. 5,139,941 and 6,376,237.

The term “accessory functions” refers to non-AAV derived viral and/or cellular functions upon which AAV is dependent for its replication. Thus, the term captures proteins and RNAs that are required in AAV replication, including those moieties involved in activation of AAV gene transcription, stage specific AAV mRNA splicing, AAV DNA replication, synthesis of Cap expression products and AAV capsid assembly. Viral-based accessory functions can be derived from any of the known helper viruses, such as adenovirus, herpesvirus (other than herpes simplex virus type-1) and vaccinia virus.

The term “accessory function vector” refers generally to a nucleic acid molecule that includes nucleotide sequences providing accessory functions. An accessory function vector can be transfected into a suitable host cell, wherein the vector is then capable of supporting AAV virion production in the host cell. Expressly excluded from the term are infectious viral particles as they exist in nature, such as adenovirus, herpesvirus or vaccinia virus particles. Thus, accessory function vectors can be in the form of a plasmid, phage, transposon or cosmid.

In particular, it has been demonstrated that the full-complement of adenovirus genes are not required for accessory helper functions. In particular, adenovirus mutants incapable of DNA replication and late gene synthesis have been shown to be permissive for AAV replication. Ito et al., (1970) J. Gen. Virol. 9:243; Ishibashi et al, (1971) Virology 45:317. Similarly, mutants within the E2B and E3 regions have been shown to support AAV replication, indicating that the E2B and E3 regions are probably not involved in providing accessory functions. Carter et al., (1983) Virology 126:505. However, adenoviruses defective in the E1 region, or having a deleted E4 region, are unable to support AAV replication. Thus, E1A and E4 regions are likely required for AAV replication, either directly or indirectly. Laughlin et al., (1982) J. Virol. 41:868; Janik et al., (1981) Proc. Natl. Acad. Sci. USA 78:1925; Carter et al., (1983) Virology 126:505. Other characterized Ad mutants include: E1B (Laughlin et al. (1982), supra; Janik et al. (1981), supra; Ostrove et al., (1980) Virology 104:502); E2A (Handa et al., (1975) J. Gen. Virol. 29:239; Strauss et al., (1976) J. Virol. 17:140; Myers et al., (1980) J. Virol. 35:665; Jay et al., (1981) Proc. Natl. Acad. Sci. USA 78:2927; Myers et al., (1981) J. Biol. Chem. 256:567); E2B (Carter, Adeno-Associated Virus Helper Functions, in I CRC Handbook of Parvoviruses (P. Tijssen ed., 1990)); E3 (Carter et al. (1983), supra); and E4 (Carter et al. (1983), supra; Carter (1995)). Although studies of the accessory functions provided by adenoviruses having mutations in the E1B coding region have produced conflicting results, Samulski et al., (1988) J. Virol. 62:206-210, recently reported that E1B55k is required for AAV virion production, while E1B19k is not. In addition, International Publication WO 97/17458 and Matshushita et al., (1998) Gene Therapy 5:938-945, describe accessory function vectors encoding various Ad genes. Particularly preferred accessory function vectors comprise an adenovirus VA RNA coding region, an adenovirus E4 ORF6 coding region, an adenovirus E2A 72 kD coding region, an adenovirus E1A coding region, and an adenovirus E1B region lacking an intact E1B55k coding region. Such vectors are described in International Publication No. WO 01/83797.

By “recombinant virus” is meant a virus that has been genetically altered, e.g., by the addition or insertion of a heterologous nucleic acid construct into the particle.

By “AAV virion” is meant a complete virus particle, such as a wild-type (wt) AAV virus particle (comprising a linear, single-stranded AAV nucleic acid genome associated with an AAV capsid protein coat). In this regard, single-stranded AAV nucleic acid molecules of either complementary sense, e.g., “sense” or “antisense” strands, can be packaged into any one AAV virion and both strands are equally infectious.

A “recombinant AAV virion,” or “rAAV virion” is defined herein as an infectious, replication-defective virus including an AAV protein shell, encapsidating a heterologous nucleotide sequence of interest which is flanked on both sides by AAV ITRs. A rAAV virion is produced in a suitable host cell which has had an AAV vector, AAV helper functions and accessory functions introduced therein. In this manner, the host cell is rendered capable of encoding AAV polypeptides that are required for packaging the AAV vector (containing a recombinant nucleotide sequence of interest) into infectious recombinant virion particles for subsequent gene delivery.

The term “empty capsid” refers to a recombinant AAV that includes an AAV protein shell but that lacks in whole or part the polynucleotide construct comprising the heterologous nucleotide sequence of interest flanked on both sides by AAV ITRs. Accordingly, the empty capsid does not efficiently function to transfer the gene of interest into the host cell.

The term “transfection” is used to refer to the uptake of foreign DNA by a cell, and a cell has been “transfected” when exogenous DNA has been introduced inside the cell membrane. A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous DNA moieties, such as a nucleotide integration vector and other nucleic acid molecules, into suitable host cells.

The term “host cell” denotes, for example, microorganisms, yeast cells, insect cells, and mammalian cells, that can be, or have been, used as recipients of an AAV helper construct, an AAV vector plasmid, an accessory function vector, or other transfer DNA. The term includes the progeny of the original cell which has been transfected. Thus, a “host cell” as used herein generally refers to a cell which has been transfected with an exogenous DNA sequence. It is understood that the progeny of a single parental cell may not necessarily be completely identical in morphology or in genomic or total DNA complement as the original parent, due to natural, accidental, or deliberate mutation.

As used herein, the term “cell line” refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants.

A stock of AAV virions comprising packaged genomes is “substantially free of” empty capsids when at least about 60%-99% or more of the virions present in the stock are rAAV virions with packaged genomes. Preferably, the packaged genomes comprise at least about 75% to 85%, more preferably about 90% of the virions present in the stock, even more preferably at least about 95%, or even 99% or more by weight of the virions present in the stock, or any integer between these ranges. Thus, a stock is substantially free of empty capsids when from about 40% to about 1% or less, preferably about 25% to about 15% or less, more preferably about 10% or less, even more preferably about 5% to about 1% or less of the resulting stock comprises empty capsids.

A “nucleic acid” sequence refers to a DNA or RNA sequence. The term captures sequences that include any of the known base analogues of DNA and RNA such as, but not limited to 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, -uracil-5-oxyacetic acid methylester, uracil-5-oxyacetic acid, pseudouracil, queosine, 2-thiocytosine, and 2,6-diaminopurine.

The term DNA “control sequences” refers collectively to promoter sequences, polyadenylation signals, transcription termination sequences, upstream regulatory domains, origins of replication, internal ribosome entry sites (“IRES”), enhancers, and the like, which collectively provide for the replication, transcription and translation of a coding sequence in a recipient cell. Not all of these control sequences need always be present so long as the selected coding sequence. is capable of being replicated, transcribed and translated in an appropriate host cell.

The term “promoter” is used herein in its ordinary sense to refer to a nucleotide region comprising a DNA regulatory sequence, wherein the regulatory sequence is derived from a gene which is capable of binding RNA polymerase and initiating transcription of a downstream (3′-direction) coding sequence. Transcription promoters can include “inducible promoters” (where expression of a polynucleotide sequence operably linked to the promoter is induced by an analyte, cofactor, regulatory protein, etc.), “repressible promoters” (where expression of a polynucleotide sequence operably linked to the promoter is induced by an analyte, cofactor, regulatory protein, etc.), and “constitutive promoters”.

“Operably linked” refers to an arrangement of elements wherein the components so described are configured so as to perform their usual function. Thus, control sequences operably linked to a coding sequence are capable of effecting the expression of the coding sequence. The control sequences need not be contiguous with the coding sequence, so long as they function to direct the expression thereof. Thus, for example, intervening untranslated yet transcribed sequences can be present between a promoter sequence and the coding sequence and the promoter sequence can still be considered “operably linked” to the coding sequence.

For the purpose of describing the relative position of nucleotide sequences in a particular nucleic acid molecule throughout the instant application, such as when a particular nucleotide sequence is described as being situated “upstream,” “downstream,” “3′,” or “5′” relative to another sequence, it is to be understood that it is the position of the sequences in the “sense” or “coding” strand of a DNA molecule that is being referred to as is conventional in the art.

A “functional homologue,” or a “functional equivalent” of a given AAV polypeptide includes molecules derived from the native polypeptide sequence, as well as recombinantly produced or chemically synthesized polypeptides which function in a manner similar to the reference AAV molecule to achieve a desired result. Thus, a functional homologue of AAV Rep68 or Rep78 encompasses derivatives and analogues of those polypeptides—including any single or multiple amino acid additions, substitutions and/or deletions occurring internally or at the amino or carboxy termini thereof—so long as integration activity remains.

A “functional homologue,” or a “functional equivalent” of a given adenoviral nucleotide region includes similar regions derived from a heterologous adenovirus serotype, nucleotide regions derived from another virus or from a cellular source, as well as recombinantly produced or chemically synthesized polynucleotides which function in a manner similar to the reference nucleotide region to achieve a desired result. Thus, a functional homologue of an adenoviral VA RNA gene region or an adenoviral E2a gene region encompasses derivatives and analogues of such gene regions—including any single or multiple nucleotide base additions, substitutions and/or deletions occurring within the regions, so long as the homologue retains the ability to provide its inherent accessory function to support AAV virion production at levels detectable above background.

B. General Methods

The present invention involves reducing the numbers of, or eliminating, empty capsids contained within purified stocks of AAV virions, with minimal loss to packaged capsids contained therein. The methods of the present invention may be used regardless of the process in which rAAV virions are produced.

There are several methods that are well known in the art for generating rAAV virions: for example, co-infection with one of the AAV helper viruses (e.g., adenovirus, herpesvirus, or vaccinia virus) or transfection with a recombinant AAV vector, an AAV helper vector, and an accessory function vector. For detailed descriptions of methods for generating rAAV virions see, U.S. Pat. Nos. 6,001,650 and 6,004,797, both incorporated herein by reference in their entireties.

For example, wild-type AAV and helper viruses may be used to provide the necessary replicative functions for producing rAAV virions (see, e.g., U.S. Pat. No. 5,139,941, incorporated herein by reference in its entirety). Alternatively, a plasmid, containing helper function genes, in combination with infection by one of the well-known helper viruses can be used as the source of replicative functions (see e.g., U.S. Pat. No. 5,622,856 and U.S. Pat. No. 5,139,941, both incorporated herein by reference in their entireties). Similarly, a plasmid, containing accessory function genes can be used in combination with infection by wild-type AAV, to provide the necessary replicative functions. These three approaches, when used in combination with a rAAV vector, are each sufficient to produce rAAV virions. Other approaches, well known in the art, can also be employed by the skilled artisan to produce rAAV virions.

In a preferred embodiment of the present invention, a triple transfection method (described in detail in U.S. Pat. No. 6,001,650, incorporated by reference herein in its entirety) is used to produce rAAV virions because this method does not require the use of an infectious helper virus, enabling rAAV virions to be produced without any detectable helper virus present. This is accomplished by use of three vectors for rAAV virion production: an AAV helper function vector, an accessory function vector, and a rAAV expression vector. One of skill in the art will appreciate, however, that the nucleic acid sequences encoded by these vectors can be provided on two or more vectors in various combinations.

As explained herein, the AAV helper function vector encodes the “AAV helper function” sequences (i.e., rep and cap), which function in trans for productive AAV replication and encapsidation. Preferably, the AAV helper function vector supports efficient AAV vector production without generating any detectable wt AAV virions (i.e., AAV virions containing functional rep and cap genes). An example of such a vector, pHLP19 is described in U.S. Pat. No. 6,001,650, incorporated herein by reference in its entirety. The rep and cap genes of the AAV helper function vector can be derived from any of the known AAV serotypes, as explained above. For example, the AAV helper function vector may have a rep gene derived from AAV-2 and a cap gene derived from AAV-6; one of skill in the art will recognize that other rep and cap gene combinations are possible, the defining feature being the ability to support rAAV virion production.

The accessory function vector encodes nucleotide sequences for non-AAV derived viral and/or cellular functions upon which AAV is dependent for replication (i.e., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the well-known helper viruses such as adenovirus, herpesvirus (other than herpes simplex virus type-1), and vaccinia virus. In a preferred embodiment, the accessory function plasmid pLadeno5 is used (details regarding pLadeno5 are described in U.S. Pat. No. 6,004,797, incorporated herein by reference in its entirety). This plasmid provides a complete set of adenovirus accessory functions for AAV vector production, but lacks the components necessary to form replication-competent adenovirus.

Once stocks of AAV virions are produced, a number of methods, detailed below, can be used to determine infectious titers and to purify packaged genomes away from empty capsids.

In order to further an understanding of the invention, a more detailed discussion is provided below regarding recombinant AAV expression vectors, AAV helper and accessory functions, compositions comprising AAV virions, as well as delivery of virions.

Recombinant AAV Expression Vectors

Recombinant AAV (rAAV) expression vectors are constructed using known techniques to at least provide as operatively linked components in the direction of transcription, control elements including a transcriptional initiation region, the polynucleotide of interest and a transcriptional termination region. The control elements are selected to be functional in a mammalian muscle cell. The resulting construct which contains the operatively linked components is bounded (5′ and 3′) with functional AAV ITR sequences.

The nucleotide sequences of AAV ITR regions are known. See, e.g., Kotin, R. M. (1994) Human Gene Therapy 5:793-801; Berns, K. I. “Parvoviridae and their Replication” in Fundamental Virology, 2nd Edition, (B. N. Fields and D. M. Knipe, eds.) for the AAV-2 sequence. AAV ITRs used in the vectors of the invention need not have a wild-type nucleotide sequence, and may be altered, e.g., by the insertion, deletion or substitution of nucleotides. Additionally, AAV ITRs may be derived from any of several AAV serotypes, including without limitation, AAV-1, AAV-2, AAV-3, AAV-4, AAV-5, AAV-6, AAV-7 and AAV-8, etc. Furthermore, 5′ and 3′ ITRs which flank a selected nucleotide sequence in an AAV expression vector need not necessarily be identical or derived from the same AAV serotype or isolate, so long as they function as intended, i.e., to allow for excision and rescue of the sequence of interest from a host cell genome or vector, and to allow integration of the DNA molecule into the recipient cell genome when AAV Rep gene products are present in the cell.

Suitable polynucleotide molecules for use in AAV vectors will be less than about 5 kilobases (kb) in size. The selected polynucleotide sequence is operably linked to control elements that direct the transcription or expression thereof in the subject in vivo. Such control elements can comprise control sequences normally associated with the selected gene. Alternatively, heterologous control sequences can be employed. Useful heterologous control sequences generally include those derived from sequences encoding mammalian or viral genes. Examples include, but are not limited to, neuron-specific enolase promoter, a GFAP promoter, the SV40 early promoter, mouse mammary tumor virus LTR promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, synthetic promoters, hybrid promoters, and the like. In addition, sequences derived from nonviral genes, such as the murine metallothionein gene, will also find use herein. Such promoter sequences are commercially available from, e.g., Stratagene (San Diego, Calif.).

The AAV expression vector which harbors the polynucleotide molecule of interest bounded by AAV ITRs, can be constructed by directly inserting the selected sequence(s) into an AAV genome which has had the major AAV open reading frames (“ORFs”) excised therefrom. Other portions of the AAV genome can also be deleted, so long as a sufficient portion of the ITRs remain to allow for replication and packaging functions. Such constructs can be designed using techniques well known in the art. See, e.g., U.S. Pat. Nos. 5,173,414 and 5,139,941; International Publication Nos. WO 92/01070 (published Jan. 23, 1992) and WO 93/03769 (published Mar. 4, 1993); Lebkowski et al. (1988) Molec. Cell. Biol. 8:3988-3996; Vincent et al. (1990) Vaccines 90 (Cold Spring Harbor Laboratory Press); Carter (1992) Current Opinion in Biotechnology 3:533-539; Muzyczka (1992) Current Topics in Microbiol. and Immunol. 158:97-129; Kotin (1994) Human Gene Therapy 5:793-801; Shelling and Smith (1994) Gene Therapy 1:165-169; and Zhou et al. (1994) J. Exp. Med. 179:1867-1875.

Alternatively, AAV ITRs can be excised from the viral genome or from an AAV vector containing the same and fused 5′ and 3′ of a selected nucleic acid construct that is present in another vector using standard ligation techniques, such as those described in Sambrook et al., supra. For example, ligations can be accomplished in 20 mM Tris-Cl pH 7.5, 10 mM MgCl₂, 10 mM DTT, 33 μg/ml BSA, 10 mM-50 mM NaCl, and either 40 μM ATP, 0.01-0.02 (Weiss) units T4 DNA ligase at 0° C. (for “sticky end” ligation) or 1 mM ATP, 0.3-0.6 (Weiss) units T4 DNA ligase at 14° C. (for “blunt end” ligation). Intermolecular “sticky end” ligations are usually performed at 30-100 μg/ml total DNA concentrations (5-100 nM total end concentration). AAV vectors which contain ITRs have been described in, e.g., U.S. Pat. No. 5,139,941. In particular, several AAV vectors are described therein which are available from the American Type Culture Collection (“ATCC”) under Accession Numbers 53222, 53223, 53224, 53225 and 53226.

Additionally, chimeric genes can be produced synthetically to include AAV ITR sequences arranged 5′ and 3′ of one or more selected nucleic acid sequences. Preferred codons for expression of the chimeric gene sequence in mammalian muscle cells can be used. The complete chimeric sequence is assembled from overlapping oligonucleotides prepared by standard methods. See, e.g., Edge (1981) Nature 292:756; Nambair et al. (1984) Science 223:1299; Jay et al. (1984) J. Biol. Chem. (1984) 259:6311.

For the purposes of the invention, suitable host cells for producing rAAV virions from the AAV expression vectors include microorganisms, yeast cells, insect cells, and mammalian cells, that can be, or have been, used as recipients of a heterologous DNA molecule and that are capable of growth in suspension culture. The term includes the progeny of the original cell which has been transfected. Thus, a “host cell” as used herein generally refers to a cell which has been transfected with an exogenous DNA sequence. Cells from the stable human cell line, 293 (readily available through, e.g., the American Type Culture Collection under Accession Number ATCC CRL1573) are preferred in the practice of the present invention. Particularly, the human cell line 293 is a human embryonic kidney cell line that has been transformed with adenovirus type-5 DNA fragments (Graham et al. (1977) J. Gen. Virol. 36:59), and expresses the adenoviral E1a and E1b genes (Aiello et al. (1979) Virology 94:460). The 293 cell line is readily transfected, and provides a particularly convenient platform in which to produce rAAV virions.

AAV Helper Functions

Host cells containing the above-described AAV expression vectors must be rendered capable of providing AAV helper functions in order to replicate and encapsidate the nucleotide sequences flanked by the AAV ITRs to produce rAAV virions. AAV helper functions are generally AAV-derived coding sequences which can be expressed to provide AAV gene products that, in turn, function in trans for productive AAV replication. AAV helper functions are used herein to complement necessary AAV functions that are missing from the AAV expression vectors. Thus, AAV helper functions include one, or both of the major AAV ORFs, namely the rep and cap coding regions, or functional homologues thereof.

By “AAV rep coding region” is meant the art-recognized region of the AAV genome which encodes the replication proteins Rep 78, Rep 68, Rep 52 and Rep 40. These Rep expression products have been shown to possess many functions, including recognition, binding and nicking of the AAV origin of DNA replication, DNA helicase activity and modulation of transcription from AAV (or other heterologous) promoters. The Rep expression products are collectively required for replicating the AAV genome. For a description of the AAV rep coding region, see, e.g., Muzyczka, N. (1992) Current Topics in Microbiol. and Immunol. 158:97-129; and Kotin, R. M. (1994) Human Gene Therapy 5:793-801. Suitable homologues of the AAV rep coding region include the human herpesvirus 6 (HHV-6) rep gene which is also known to mediate AAV-2 DNA replication (Thomson et al. (1994) Virology 204:304-311).

By “AAV cap coding region” is meant the art-recognized region of the AAV genome which encodes the capsid proteins VP1, VP2, and VP3, or functional homologues thereof. These Cap expression products supply the packaging functions which are collectively required for packaging the viral genome. For a description of the AAV cap coding region, see, e.g., Muzyczka, N. and Kotin, R. M. (supra).

AAV helper functions are introduced into the host cell by transfecting the host cell with an AAV helper construct either prior to, or concurrently with, the transfection of the AAV expression vector. AAV helper constructs are thus used to provide at least transient expression of AAV rep and/or cap genes to complement missing AAV functions that are necessary for productive AAV infection. AAV helper constructs lack AAV ITRs and can neither replicate nor package themselves.

These constructs can be in the form of a plasmid, phage, transposon, cosmid, virus, or virion. A number of AAV helper constructs have been described, such as the commonly used plasmids pAAV/Ad and pIM29+45 which encode both Rep and Cap expression products. See, e.g., Samulski et al. (1989) J. Virol. 63:3822-3828; and McCarty et al. (1991) J. Virol. 65:2936-2945. A number of other vectors have been described which encode Rep and/or Cap expression products. See, e.g., U.S. Pat. No. 5,139,941.

Both AAV expression vectors and AAV helper constructs can be constructed to contain one or more optional selectable markers. Suitable markers include genes which confer antibiotic resistance or sensitivity to, impart color to, or change the antigenic characteristics of those cells which have been transfected with a nucleic acid construct containing the selectable marker when the cells are grown in an appropriate selective medium. Several selectable marker genes that are useful in the practice of the invention include the hygromycin B resistance gene (encoding Aminoglycoside phosphotranferase (APH)) that allows selection in mammalian cells by conferring resistance to G418 (available from Sigma, St. Louis, Mo.). Other suitable markers are known to those of skill in the art.

AAV Accessory Functions

The host cell (or packaging cell) must also be rendered capable of providing nonAAV-derived functions, or “accessory functions,” in order to produce rAAV virions. Accessory functions are nonAAV-derived viral and/or cellular functions upon which AAV is dependent for its replication. Thus, accessory functions include at least those nonAAV proteins and RNAs that are required in AAV replication, including those involved in activation of AAV gene transcription, stage specific AAV mRNA splicing, AAV DNA replication, synthesis of Cap expression products and AAV capsid assembly. Viral-based accessory functions can be derived from any of the known helper viruses.

In particular, accessory functions can be introduced into and then expressed in host cells using methods known to those of skill in the art. Typically, accessory functions are provided by infection of the host cells with an unrelated helper virus. A number of suitable helper viruses are known, including adenoviruses; herpesviruses such as herpes simplex virus types 1 and 2; and vaccinia viruses. Nonviral accessory functions will also find use herein, such as those provided by cell synchronization using any of various known agents. See, e.g., Buller et al. (1981) J. Virol. 40:241-247; McPherson et al. (1985) Virology 147:217-222; Schlehofer et al. (1986) Virology 152:110-117.

Alternatively, accessory functions can be provided using an accessory function vector as defined above. See, e.g., U.S. Pat. No. 6,004,797 and International Publication No. WO 01/83797, incorporated herein by reference in its entirety. Nucleic acid sequences providing the accessory functions can be obtained from natural sources, such as from the genome of an adenovirus particle, or constructed using recombinant or synthetic methods known in the art. As explained above, it has been demonstrated that the full-complement of adenovirus genes are not required for accessory helper functions. In particular, adenovirus mutants incapable of DNA replication and late gene synthesis have been shown to be permissive for AAV replication. Ito et al., (1970) J. Gen. Virol. 9:243; Ishibashi et al, (1971) Virology 45:317. Similarly, mutants within the E2B and E3 regions have been shown to support AAV replication, indicating that the E2B and E3 regions are probably not involved in providing accessory functions. Carter et al., (1983) Virology 126:505. However, adenoviruses defective in the E1 region, or having a deleted E4 region, are unable to support AAV replication. Thus, E1A and E4 regions are likely required for AAV replication, either directly or indirectly. Laughlin et al., (1982) J. Virol. 41:868; Janik et al., (1981) Proc. Natl. Acad. Sci. USA 78:1925; Carter et al., (1983) Virology 126:505. Other characterized Ad mutants include: E1B (Laughlin et al. (1982), supra; Janik et al. (1981), supra; Ostrove et al., (1980) Virology 104:502); E2A (Handa et al., (1975) J. Gen. Virol. 29:239; Strauss et al., (1976) J. Virol. 17:140; Myers et al., (1980) J. Virol. 35:665; Jay et al., (1981) Proc. Natl. Acad. Sci. USA 78:2927; Myers et al., (1981) J. Biol. Chem. 256:567); E2B (Carter, Adeno-Associated Virus Helper Functions, in I CRC Handbook of Parvoviruses (P. Tijssen ed., 1990)); E3 (Carter et al. (1983), supra); and E4 (Carter et al.(1983), supra; Carter (1995)). Although studies of the accessory functions provided by adenoviruses having mutations in the E1B coding region have produced conflicting results, Samulski et al., (1988) J. Virol. 62:206-210, recently reported that E1B55k is required for AAV virion production, while E1B19k is not. In addition, International Publication WO 97/17458 and Matshushita et al., (1998) Gene Therapy 5:938-945, describe accessory function vectors encoding various Ad genes. Particularly preferred accessory function vectors comprise an adenovirus VA RNA coding region, an adenovirus E4 ORF6 coding region, an adenovirus E2A 72 kD coding region, an adenovirus E1A coding region, and an adenovirus E1B region lacking an intact E1B55k coding region. Such vectors are described in International Publication No. WO 01/83797.

As a consequence of the infection of the host cell with a helper virus, or transfection of the host cell with an accessory function vector, accessory functions are expressed which transactivate the AAV helper construct to produce AAV Rep and/or Cap proteins. The Rep expression products excise the recombinant DNA (including the DNA of interest) from the AAV expression vector. The Rep proteins also serve to duplicate the AAV genome. The expressed Cap proteins assemble into capsids, and the recombinant AAV genome is packaged into the capsids. Thus, productive AAV replication ensues, and the DNA is packaged into rAAV virions.

Purification of rAAV Virions

Following recombinant AAV replication, rAAV virions can be purified from the host cell using a variety of conventional purification methods, such as column chromatography, CsCl gradients, and the like. For example, a plurality of column purification steps can be used, such as purification over an anion exchange column, an affinity column and/or a cation exchange column. See, for example, International Publication No. WO 02/12455. Further, if infection is employed to express the accessory functions, residual helper virus can be inactivated, using known methods. For example, adenovirus can be inactivated by heating to temperatures of approximately 60° C. for, e.g., 20 minutes or more. This treatment effectively inactivates only the helper virus since AAV is extremely heat stable while the helper adenovirus is heat labile.

Recombinant AAV vectors containing any number of reporter genes can be used to determine infectious titers using the methods of the disclosed invention. For example, alkaline phosphatase, β-galactosidase (LacZ), green fluorescent protein, or luciferase is contemplated for use in the invention. After harvesting the transfected host cell, a lysate is formed by disrupting the transfected host cells using techniques suitable for large-scale production, such as microfluidization. The lysate is then filtered (for example, through a 0.45 μm filter), and purified using column chromatographic methods (for example, pouring the filtered lysate over a POROS HE column).

The purified AAV stock (e.g., a lysate produced as described above) is then treated to remove empty capsids. For example, the purified AAV stock can be treated with destabilizing agents such as heat, urea, and chemicals that cause changes in pH. Samples are preferably treated with 0.5 to 2% SDS, most preferably 1% SDS (final concentration after addition), or any value within these ranges, and then heated to temperatures from about 40-70° C., such as 45-65° C., 45-60° C., 50-70° C., 55-65° C., 60° C., or any integer within these ranges, for 2 minutes to 30 minutes, preferably 3 minutes to 20 minutes, even more preferably 4 minutes to 10 minutes, or any integer within these ranges, such as 4.3, 4.4, 4.5 . . . 5 . . . 5.2, 5.3 . . . minutes. Alternatively, samples can be heated first, or heated and treated with destabilizing agents substantially concurrently. Samples may also be treated with urea, preferably at final concentrations from about 4 to 5M, more preferably from about 4 to 4.5M and most preferably about 4M, and the pH of the samples can be adjusted to levels from between 4 to 7, preferably 4.5 to 6.0, more preferably from about 4.5 to 5.5, and most preferably to about 4.5 to 5, such as 5.0. Chemicals such as acetic acid, formic acid, citric acid, 2Morpholinoethanesulfonic acid monohydrate (MES), 4(2Hydroxyethyl)piperazine-1ethanesulfonic acid HEPES, 2-(N-Cyclohexylamino)ethanesulfonic acid (CHES), and 3-(Cyclohexylamino)-1-propanesulfonic acid (CAPS), as well as others that are well known in the art may be used to adjust pH levels. MES, HEPES, CHES, and CAPS are available from commercial vendors such as Sigma-Aldrich, Inc., St. Louis, Mo.

Methods for assaying for empty capsids and rAAV virions with packaged genomes are known in the art. See, e.g., Grimm et al., Gene Therapy (1999) 6:1322-1330. To test for denatured capsid, the methods include subjecting the treated AAV stock to SDS-polyacrylamide get electrophoresis, consisting of any gel capable of separating the three capsid proteins, for example, a gradient gel containing 3-8% Tris-acetate in the buffer, then running the gel until sample material is separated, and blotting the gel onto nylon or nitrocellulose membranes, preferably nylon. Anti-AAV capsid antibodies are then used as the primary antibodies that bind to denatured capsid proteins, preferably an anti-AAV capsid monoclonal antibody, most preferably the B1 anti-AAV-2 monoclonal antibody (Wobus et al., J. Virol. (2000) 74:9281-9293). A secondary antibody is then used, one that binds to the primary antibody and contains a means for detecting binding with the primary antibody, more preferably an anti-IgG antibody containing a detection molecule covalently bound to it, most preferably a sheep anti-mouse IgG antibody covalently linked to horseradish peroxidase. A method for detecting binding is used to semi-quantitatively determine binding between the primary and secondary antibodies, preferably a detection method capable of detecting radioactive isotope emissions, electromagnetic radiation, or calorimetric changes, most preferably a chemiluminescence detection kit.

To test for infectious titer, the methods include the seeding of about 100,000 host cells, preferably of human origin, most preferably HeLa cells, into tissue culture-treated plates, preferably 24-well tissue culture-treated plates, and incubated for about 24 hours after which adenovirus, preferably the adenovirus-2 serotype, and treated rAAV stock is added to the host cells. The host cells, adenovirus, and rAAV stock are allowed to incubate for 24 hours, after which the host cells are fixed, preferably with formaldehyde and glutaraldehyde, and stained with an appropriate agent that will detect the rAAV expressed transgene; for example, with rAAV-LacZ, X-gal is contemplated as the staining agent. Other agents for other reporter genes are well known in the art.

C. Experimental

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### EXAMPLE 1

### Construction of the Recombinant AAV Plasmid pVmLacZ

A 4311 bp XbaI DNA fragment was excised from pSUB201 (Samulski et al. (1987) J. Virol 61:3096-3101) which contains AAV rep/cap sequences. The XbaI ends were reannealed with a 10 bp NotI synthetic oligo (“AGCGGCCGCT”) to give a plasmid intermediate pUC/ITR-NotI that has both AAV ITR's separated by 116 bp of residual AAV sequence and NotI linker DNA.

A 1319 bp NotI DNA fragment was excised from plasmid p1.1c containing CMV promoter and hGH intron sequences. This DNA sequence was inserted into the NotI site of pUC/ITR-Not I, to give the intermediate pSUB201N.

A 1668 bp PvuII (5131-1493) ITR bound CMV expression cassette was excised from pSUB201N and inserted at the PvuII site (position 12) of pWee.1a (see, U.S. Pat. No. 6,309,634, incorporated herein by reference in its entirety), to give the plasmid intermediate pWee.1b. The excision of the 1668bp PvuII fragment from pSUB201N removed 15 bp from the outside of each ITR, in the “A” palindromic region.

A 4737 bp Not I/EcoRV “AAVrep/cap” DNA sequence was excised from pGN1909 (U.S. Pat. No. 5,622,856, incorporated herein by reference in its entirety) and the ends were rendered blunt by filling in the 3′ recesses using Klenow DNA polymerase. AscI linkers were ligated to both ends, followed by cloning this “pGN1909/AscI” DNA fragment into the backbone of pWee.1b at an AscI site (2703), to give the intermediate pWee1909 (8188 bp). This plasmid has the ITR-bound CMV expression cassette with an AAV rep/cap gene backbone.

A 3246 bp SmaI/DraI LacZ gene was excised from pCMV-beta (Clonetech) and AscI linkers were ligated to the blunt-ended fragment. This LacZ/AscI fragment was cloned into p1.1c between BssHII sites, to give p1.1cADHLacZ, that has the LacZ gene driven by the CMV promoter.

A 4387 bp NotI DNA fragment was excised from p1.1cADHLacZ, that has the LacZ gene driven by the CMV promoter. This fragment was inserted between the NotI sites of pWee1909, after removing a 1314 bp “CMV promoter/hGH intron” expression cassette. The resulting construct, pW1909ADHLacZ, has the β-galactosidase gene under the control of the CMV promoter and bounded by ITRs. The backbone of the plasmid carries the “rep” and “cap” genes providing AAV helper functions and the β-lactamase (ampicillin) gene confers antibiotic resistance.

A 4772 bp SseI DNA fragment containing a “CMV/LacZ” cassette was excised from pW1909ADHLacZ and inserted into the SseI site of pUC19, to give Pre-pVLacZ. This construct still contains approximately 50 bp of remnant 5′ and 3′ pSUB201 sequences internal to each ITR.

The remnant pSUB201 sequences were removed by excising a 2912 bp MscI “pUC/ITR” DNA fragment from Pre-pVLacZ, that also removes approximately 35 bp of the “D” region of each ITR. A synthetic linker “145NA/NB(CCAACTCCATCACTAGGGGTTCCTGCGGCC)” containing an MscI restriction site, the ITR “D” region and a NotI site was used to clone in a 4384 bp NotI fragment from pW1909ADHLacZ, that has the “CMV/LacZ” expression cassette. The resulting plasmid pVLacZ, is has the β-galactosidase gene under the control of an alcohol dehydrogenase enhancer sequence and the CMV promoter, all bounded by AAV ITRs.

pVLacZ was further modified by removing LacZ elements and polylinker sequence outside of the ITR bound LacZ expression cassette as follows. A 534 bp EheI/AflIII LacZ/polylinker sequence was excised from pUC119, the ends were blunted using Klenow DNA polymerase and the plasmid was ligated to an SseI linker (CCTGCAGG), to produce pUC119/SseI. The “AAVLacZ ” DNA sequences were removed from pVLacZ by cutting out a 4666 bp SseI fragment. This SseI fragment was cloned into the SseI site of pUC119/SseI to generate pVmLacZ. pVmLacZ has the CMV promoter/ADH enhancer/β-galactosidase gene bounded by AAV ITRs in a pUC119-derived backbone that confers ampicillin resistance and has a high copy number origin of replication.

### EXAMPLE 2

### Production and Purification of rAAV Virions

Recombinant AAV-2-lacZ virions were produced by the triple-transfection method described in U.S. Pat. Nos. 6,001,650 and 6,004,797, both incorporated herein by reference in their entireties. The plasmids used were the accessory function plasmid pladeno5, the AAV helper function plasmid pHLP19, and the recombinant AAV plasmid pVmLacZ (its construction is described in Example 1). Human embryonic kidney-293 cells (293 cells, available from ATCC, catalog number CRL-1573) were used as host cells for the production of rAAV virions.

After 72 hr post-transfection, 293 cells were disrupted by microfluidization using a Microfluidizer™ (Microfluidics International Corp., Newton, Mass.) and the crude lysate was collected and filtered through a 2.0 M ULTIPLEAT PROFILE™ filter followed by filtration through a 0.45 M SUPOR™ membrane filter (Pall Corporation, Port Washington, N.Y.). Once filtered, the clarified lysate was loaded on a POROS 20HE™ column (PerSeptive Biosystems, Inc., Framingham, Mass.). The rAAV virions were eluted with buffer containing 20 mM NaH₂PO₄ and 350 mM NaCl. The eluant was formulated in 20 mM NaH₂PO4, 150 mM NaCl, 5% sorbitol, and 0.1% Tween-80, at pH 7.4 at a concentration of 4×10¹² vector genomes/milliliter (vg/mL).

### EXAMPLE 3

### Recombinant AAV Stock Treatment and Western Blots

3A. Recombinant AAV stocks purified as in Example 2 were diluted in 1% SDS and 4M urea and then subjected to changes of temperature and pH. Various samples of AAV stocks were treated with pH-altering chemicals (875 mM vinegar, 100 mM citric acid, 100 mM acetate, 100 mM MES, 100 mM HEPES, 100 mM CHES, and 100 mM CAPS were used as the pH-adjusting agents) and heated for 5 min, then stored on ice prior to Western blotting. Western blots were carried out using standard techniques, except that samples were not boiled prior to loading. Briefly, treated AAV stock samples were loaded onto 3-8% Tris-acetate polyacrylamide gels having a pH of about 7 (Invitrogen, San Diego, Calif.) and electrophoresed in a XCell Sure Lock gel apparatus (Invitrogen, San Diego, Calif.) at 75 milliamps for about 1 hr, allowing for the loading dye front to reach the bottom of the gel. After electrophoresis was complete, the gel was rinsed in transfer buffer, samples electrotransferred at 100 V for 1 hr to a nylon membrane, and the membrane placed in a container containing blocking solution containing PBS with 3% BSA and placed on a shaker for 1 hr at room temperature. After incubation with blocking solution, the blocking solution was discarded. The membrane was placed in a container with primary antibody (B1 anti-AAV-2 monoclonal antibody, Wobus et al., J. Virol. (2000) 74:9281-9293, diluted in PBS containing 3% BSA at a concentration of between 1:10 and 1:20 primary antibody:blocking solution, Maine Biotechnology Services, Portland, Me.) that was diluted in blocking solution, and the container was agitated for 1 hr at room temperature. After incubation with the primary antibody, the membrane was washed with PBS containing 0.3% Igepal CA-630 detergent (Sigma-Aldrich, Inc., St. Louis, Mo.) for 10 min wash at room temperature under agitation and then the wash step repeated twice. After the third wash, the membrane was incubated with the secondary antibody at a dilution of 1:12000 secondary antibody:PBS containing 1% BSA and 0.1% CA-630 (sheep anti-mouse IgG coupled to horseradish peroxidase, Amersham Pharmacia Biotech, Piscataway, N.J.) diluted in blocking solution and agitated for 1 hr at room temperature. After incubation with secondary antibody, the secondary antibody solution was discarded, and the membrane washed three times with PBS containing 0.3% CA-630 detergent, as before. Detection of horseradish peroxidase was by the ECL Plus chemiluminescence kit (Amersham Pharmacia Biotech, Piscataway, N.J.) following manufacturer's instructions.

Conditions of low and high pH (2.4-3.0, 9.0-11.0) resulted in complete denaturation of viral capsid (empty and packaged), as did high temperatures (>55° C.). The dark band on the lowest part of the photograph represents denatured AAV capsids. In the pH range of about 4.5-5.0 and a temperature range of about 40-55° C., however, empty capsids are denatured while many packaged capsids are left intact.

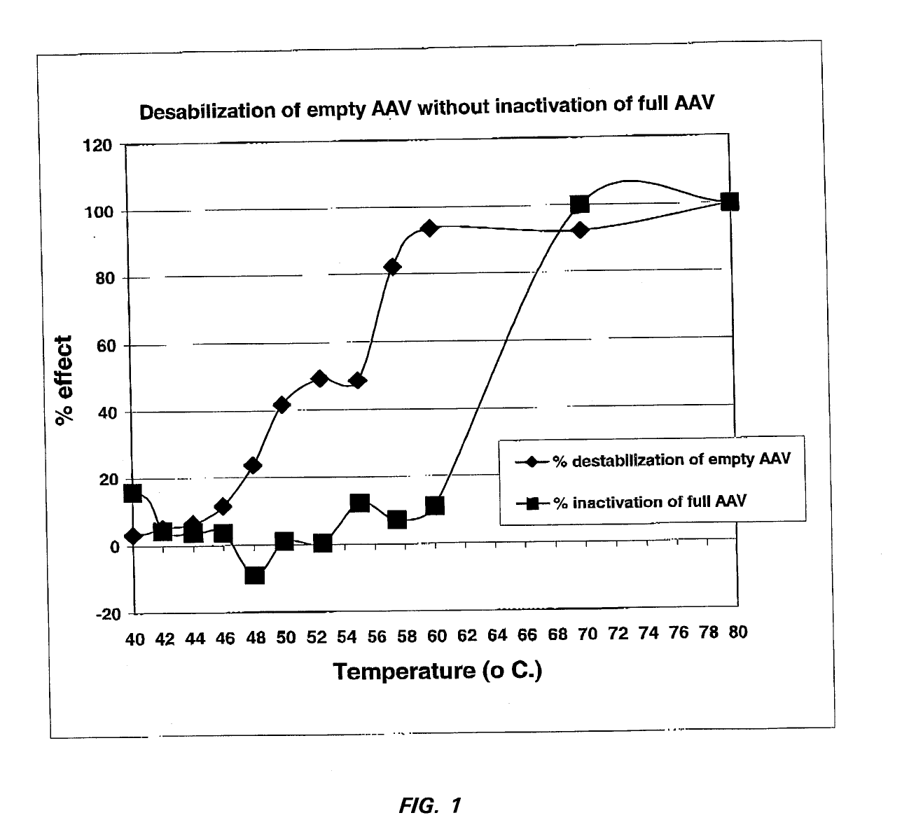

3B. Another experiment was conducted where recombinant AAV stocks purified as in Example 2 were diluted in 30 mM sodium acetate, pH 4.8, 4M urea and 1% SDS and then subjected to changes of temperature in the range of 40-80° C., heated for 5 min, then stored on ice prior to Western blotting. Western blots were carried out as described above. Western blots were scanned, quantitated, and the results are shown in FIG. 1. As shown in the figure, under these conditions, denaturation of empty capsids began at about 45° C. and was complete at about 60° C. However, the biological activity of full capsids was reduced no more than 10% over this temperature range. In fact, temperatures over 70° C. were required to complete inactivation of full capsids.

### EXAMPLE 4

### Recombinant AAV Infectious Titers

4A. HeLa cells were seeded onto 24-well plates at a concentration of 1×10⁵ cells per well. HeLa cells were infected with both wild-type adenovirus serotype 2 and treated rAAV samples (i.e., samples described in Example 3A) at 24 hr post-seeding. HeLa cells were fixed with 2.0% formaldehyde and 0.2% glutaraldehyde in PBS and stained overnight at 37° C. with 1 mg/mL X-gal 24 hr post-infection, according to the methods disclosed in U.S. Pat. No. 6,218,180, herein incorporated by reference. Blue cells were counted using a light microscope. Infectious titers were evident at a range of pH values, from pH 4.5 to pH of 9.0 (at 40° C.). A pH value of 4.0 or less (e.g., pH=3.0) resulted in complete denaturation of all viral capsids. Similarly, complete viral capsid denaturation was observed for pH values greater than 9.0. Regarding temperature, packaged capsids appeared to be stable from a range of 40-60° C., but over a relatively narrow pH range of 5.0-7.0. Empty capsids, however, appeared to be much less stable than packaged capsids over the same temperature and pH ranges (see especially, for example pH 5.0 and temperatures 40-55° C.).

4B. The experiment described in Example 4A was also conducted using the samples treated as in Example 3B. Results are shown in FIG. 1. Infectious titers were evident over a range of temperatures from about 45-60° C. Thus, for example, at 60° C., the empty capsids were completely denatured while full capsids were about 90% intact. The final conditions of treatment were very important for achieving selective destabilization of empty capsids relative to full capsids. For example, the presence of phosphate decreased the stability of both empty and full capsids and diminished the difference in stability between them (data not shown). Calcium increased the stability of both empty and full capsids and diminished the difference in stability between them (data not shown). The optimal pH was between about 4.5 and 5.0. Below 4.0 or above 5.5 (in 1% SDS and 4M urea) empty and full capsids were denatured over about the same time.

Accordingly, novel methods for preparing stocks of rAAV virions with reduced amounts of empty capsids are provided. Although preferred embodiments of the subject invention have been described in some detail, it is understood that obvious variations can be made without departing from the spirit and the scope of the invention as defined by the appended claims.

We claim:

1. A method for removing empty adeno-associated virus (AAV) capsids from a mixture of AAV virions comprising empty and packaged AAV capsids, said method comprising:
heating said mixture to a temperature of between 40° C. and 70° C. and;
adjusting the pH value of said mixture to a pH between 4 and 7.

2. The method of claim 1, wherein said method produces a stock of rAAV virions substantially free of empty AAV capsids.

3. The method of claim 1, wherein said method produces a stock of rAAV wherein at least 75% of the AAV virions present in the stock are packaged AAV capsids.

4. The method of claim 1, wherein said method produces a stock of rAAV wherein at least 85% of the AAV virions present in the stock are packaged AAV capsids.

5. The method of claim 1, wherein said method produces a stock of rAAV wherein at least 90% of the AAV virions present in the stock are packaged AAV capsids.

6. The method of claim 1, wherein said mixture is heated between 45° C. and 65° C.

7. The method of claim 6, wherein said mixture is heated to about 60° C.

8. The method of claim 1, wherein said mixture is heated for at least 5 minutes.

9. The method of claim 6, wherein said mixture is heated for at least 5 minutes.

10. The method of claim 7, wherein said mixture is heated for at least 5 minutes.

11. The method of claim 1, wherein the pH value of said mixture is between 4.0 and 5.5.

12. The method of claim 8, wherein the pH value of said mixture is about 5.0.

13. The method of claim 1, wherein said method further comprises adding a chemical destabilizing agent to said mixture.

14. The method of claim 13, wherein said chemical destabilizing agent is sodium dodecyl sulfate.

15. The method of claim 14, wherein said sodium dodecyl sulfate is present in said mixture at a concentration of between 1% and 2%.

16. The method of claim 13, wherein said chemical destabilizing agent is urea.

17. The method of claim 16, wherein said urea is present in said mixture at a concentration of between 3 molar and 8 molar.

18. The method of claim 17, wherein said urea is present in said mixture at a concentration of between 4 molar and 5 molar.

19. The method of claim 18, wherein said urea is present in said mixture at a concentration of about 4 molar.

20. The method of claim 1, wherein heating and adjusting the pH is done substantially concurrently.

21. The method of claim 1, wherein the mixture is heated prior to adjusting the pH value.

22. The method of claim 1, wherein the pH is adjusted prior to heating said mixture.

23. The method of claim 1, wherein said mixture is from a cell lysate obtained from cells rendered capable of producing AAV virions.

24. The method of claim 23, wherein said mixture is obtained from a chromatographic column elution of the cell lysate.

25. A method for removing empty adeno-associated virus (AAV) capsids from a mixture of AAV virions comprising empty and packaged AAV capsids, said method comprising:
heating said mixture for about 5-10 minutes to a temperature of between about 55° C. and about 65° C;
adjusting the pH value of said mixture to a pH between about 4.0 and 5.5; and
adding one or more chemical destabilizing agents to said mixture.

26. The method of claim 25, wherein said one or more chemical destabilizing agents is sodium dodecyl sulfate, present in said mixture at a concentration of between 1% and 2%.

27. The method of claim 25, wherein said one or more chemical destabilizing agents is urea, present in said mixture at a concentration of between 3 molar and 8 molar.

28. The method of claim 27, wherein said urea is present in said mixture at a concentration of between 4 molar and 5 molar.

29. The method of claim 28, wherein said urea is present in said mixture at a concentration of about 4 molar.

30. The method of claim 25 wherein said one or more chemical destabilizing agents are sodium dodecyl sulfate, present in said mixture at a concentration of between 1% and 2% and urea, present in said mixture at a concentration of between 3 molar and 8 molar.

31. The method of claim 30, wherein said urea is present in said mixture at a concentration of between 4 molar and 5 molar.

32. The method of claim 31, wherein said urea is present in said mixture at a concentration of about 4 molar.

33. The method of claim 25, wherein said method produces a stock of rAAV virions substantially free of empty AAV capsids.

34. The method of claim 25, wherein said method produces a stock of rAAV wherein at least 75% of the AAV virions present in the stock are packaged AAV capsids.

35. The method of claim 25, wherein said method produces a stock of rAAV wherein at least 85% of the AAV virions present in the stock are packaged AAV capsids.

36. The method of claim 25, wherein said method produces a stock of rAAV wherein at least 90% of the AAV virions present in the stock are packaged AAV capsids.

37. The method of claim 25, wherein the mixture is heated to a temperature of about 60° C.
